(19) United States
(12) Patent Application Publication
(10) Pub. No.: US 2006/0166292 A1
(43) Pub. Date: Jul. 27, 2006

(54) Methods of diagnosing and treating prostate cancer

(76) Inventors: Patricia Berg, Accokeek, MD (US); Yan Gao Man, Burtonsville, MD (US)

Correspondence Address: JUNEAU PARTNERS, P.O. BOX 2516, ALEXANDRIA, VA, 22301, US

(21) Appl. No.: 11/338,734

(22) Filed: Jan. 25, 2006

Related U.S. Application Data
(60) Provisional application No. 60/646,636, filed on Jan. 26, 2005.

(51) Int. Cl. G01N 33/574

(52) U.S. Cl. 435/7.23

(57) ABSTRACT

The invention provides for novel diagnostic methods for detecting prostate cancer. The invention also provides methods for determining the effectiveness of prostate cancer treatment. These methods take advantage of the finding that a cellular protein termed Beta Protein 1 (BP1) is expressed at elevated levels in prostate cancer tissue and cells, and the level of expression positively correlates with the aggressiveness/invasiveness of the cancer so detected.

### GOVERNMENT INTEREST

This invention was made, in part, with Government support under contract R21 CA91149, awarded by the National Institutes of Health. The Government has certain rights in this invention.

### BACKGROUND OF THE INVENTION

1. Field of the Invention

The present invention relates to the field of diagnosis and treatment of prostate cancer. The invention further relates to diagnostic kits for detecting prostate cancer.

2. Description of the Related Art

Beta Protein 1 (BP1) is a nuclear protein that was originally isolated from the human leukemia cell line K562. Characterization of BP1 showed that it is involved in the negative regulation of the human beta globin gene promoter. Further analysis indicated that this down regulation was due to BP1 specifically binding to two regions of the beta globin gene promoter.

The BP1 cDNA has been cloned, and the gene mapped to the chromosomal location 17q21-22. Sequence analysis of the BP1 clone indicates that it is a member of the distal-less (DLX) homeotic gene family, and shares some but not all sequences, including the homeobox, with two members of this gene family, DLX7 and DLX4. The DLX7 gene has been mapped to the same chromosomal region as BP1, giving rise to the suggestion that these two proteins are isoforms; DLX4 is thought to be a third isoform. While DLX7 also binds to the negative regulatory sequences in the human beta globin gene promoter in vitro, transient transfection assays in vivo have shown that DLX7 does not down-regulate beta globin promoter. Thus, BP1 and DLX7 are distinct proteins, having at least some separable activities.

Both BP1 and DLX7 (as well as DLX4) have also previously been shown to be similarly regulated in acute myeloid leukemia (AML) and in many leukemia cell lines. This study measured BP1 mRNA levels by the RT-PCR technique. However, this technique is both expensive and time-consuming. Haga et al. did not report an inexpensive, reliable and relatively fast screen of BP1 expression using anti-BP1 antibodies.

While the Haga, et al., mention that BP1 might be a molecular marker for primitive cells and/or indicate that it is an important upstream factor in an oncogenic pathway, the significance of BP1 expression in AML and other leukemia cell lines remains unknown. The present inventors have recently shown that (BP1) RNA and protein expression are substantially increased in breast cancer cells. In Man et al., the level of BP1 protein expression was shown to be positively correlated with the aggressiveness of the cancer. However, there is no mention of elevated levels of BP1 expression in prostate cancer.

Prostate Cancer Diagnosis and Treatment

A typical screening for the diagnosis of prostate cancer is the presence of elevated levels of prostate specific antigen (PSA) in the patient's blood. Elevated levels of PSA alone (and thus a “positive” PSA test), however, is not an accurate indicator of underlying prostate cancer, since conditions such as benign prostate enlargement, inflammation, age and race may all lead to high PSA levels. Indeed, only 25-30 percent of men who have a prostate biopsy due to elevated PSA levels are found to actually have prostate cancer. Thus, upon a positive PSA test result, additional tests (such as biopsy) and/or examinations (such as a digital rectal exam) are generally required before a firm diagnosis can be made. Elevated levels of BP1 expression have not previously been associated with the detection and/or treatment of prostate cancer.

In light of the above, there is a great need in the art for cost-effective, relatively fast reacting, and sensitive molecular tools that are useful in the diagnosis and treatment of prostate cancer.

The present invention provides these heretofore needed tools, as well as methods for the detection of prostate cancer cells, including in tissue and cell samples containing a heterogeneous cell population.

### BRIEF DESCRIPTION OF THE DRAWINGS

FIG. 1. Comparison of the BP1 expression status between cancer and non-cancer cells. Paraffin-embedded human prostate tumor sections were immunostained with the anti-BP1 antibody, using the ABC method with a AP-red chromogen. Arrows identify examples of the cancer cells; Asterisks identify residual non-neoplastic acini and ducts, which have little or no BP1 expression. FIG. 1A. BP1 positive ducts are distributed as clusters. Magnification is 100×. FIG. 1B. BP1 positive and negative cells co-exist within the same duct. Magnification is 100×.

FIG. 2. BP1 expression in low- and high-grade prostate carcinomas. Paraffin-embedded human prostate tumor sections were immunostained with an anti-BP1 antibody using the ABC method with the AP-red chromogen. Arrows identify examples of the cancer cell clusters; Asterisks identify lymphocyte aggregates. All the cancer cell clusters are uniformly immunoreactive to BP1. FIG. 2A. Low grade carcinoma. Cancer cells are uniformly positive for BP1. Magnification is 100×. FIG. 2B. High-grade carcinoma. Cancer cells show the highest intensity of BP1 immunostaining. Magnification is 200×.

### SUMMARY OF THE INVENTION

The invention provides novel methods for the detection of prostate cancer. The invention also provides methods for determining the appropriate therapeutic regimens for the treatment of prostate cancer. The invention further provides methods for monitoring the effectiveness of treatment of prostate cancer over time. These novel methods are realized through the use of BP1 as a molecular marker of prostate cancer cells, whereby elevated level of BP1 expression is indicative of the presence of such cancerous cells. The invention further provides kits useful in the detection and treatment of prostate cancer.

### DETAILED DESCRIPTION OF THE INVENTION

BP1 is a gene and is a member of the homeobox gene superfamily of transcription factors that are essential for early development. Recent studies, however, revealed that BP1 mRNA was present in 80% of breast infiltrating ductal carcinoma (IDC), but it was absent in 5 of 6 matched normal controls, as measured by RT-PCR. Studies also revealed that BP1 expression was significantly higher in estrogen receptor (ER) negative that in ER positive breast tumors, 100% versus 73% (p=0.03), and in African Americans than in Caucasians, 89% versus 57% (p=0.04). Subsequent immunohistochemical studies showed that BP1 protein was barely detectable in normal reduction mammoplastics, while it was seen in 21.4%, 46%, and 81% of the hyperplastic, in situ, and invasive breast lesions, respectively. The preferential expression of BP1 in IDC, in ER negative tumors, and in African American women, if confirmed on a larger scale, could have a number of scientific and clinical implications. First, as the evolution of invasive tumors is believed to result from a progressive, multi-step process initiated and driven by a sequential accumulation of genetic and biochemical abnormalities, the elucidation of the kinetics and functions of BP1 expression during tumor progression may facilitate the identification of the precursor of invasive lesions.

Previous studies have shown that a subset of normal and hyperplastic respiratory cells with expression of heterogeneous nuclear ribonucleoprotein A2/B1, a putative transcription factor, share the same genetic abnormalities and clonal composition with their malignant counterparts.

Second, as ER negative tumors have a significantly more aggressive biological behavior and worse prognosis than ER positive tumors, BP1 expression might reliably reflect or directly contribute to the aggressiveness of breast tumors. Consequently, the development of therapeutic approaches to specifically target BP1 expression might provide a more effective treatment option for breast cancers.

Third, as the genetic composition not only determines the scope and extent, but also precede, morphologic and biochemical alterations, the comparison of the BP1 gene sequence and expression pathways among ethnic cohorts may facilitate identification of the intrinsic mechanism(s) that accounts for the significant difference in the cancer frequency among these cohorts.

Fourth, as it has been reported that immuno-therapeutic approaches targeting c-erb-B2, an oncoprotein that is over-expressed in about 20% of breast malignancies, could significantly improve prognosis, it is possible that the same approach to BP1 might generate even better results, as BP1 is over-expressed in about 80% of IDC cases. In addition, as previous studies had revealed that BP1 was over-expressed in a subset of adult leukemia cells and also in male breast cancers, it is likely that BP1 may be an important molecule that is required by multiple types of hyperplastic and neoplastic cells.

Based on the experimental data and assumptions given above, this study assessed the immunohistochemical profile of BP1 expression in prostate tumors. The main goals of this study were to determine whether a similar frequency and pattern of BP1 expression are detectable in prostate tumor tissues, and whether morphologically similar tumor cells with and without BP1 expression have different proliferation rates.

### PREFERRED EMBODIMENTS

In brief, consecutive sections of normal prostate (n=5), and prostate tumors (n=50) with co-existing normal, hyperplastic, and/or neoplastic tissues were immunohistochemically stained for BP1 and a panel of growth-related markers. The expression frequency of these molecules among cases and cell types were statistically compared.

Distinct BP1 immunostaining was seen in 20% of the normal tissue associated with malignant lesion, in 42% of the hyperplastic and in 80% of the neoplastic legions, but was barely detectable in the normal controls. Among BP1 positive cases, the number of positive cells and intensity of the immunostaining increased with the tumor progression or histologic type. In cases with co-existing normal, hyperplastic, and neoplastic tissue components, the neoplastic component consistently had the highest number of BP1 positive cells and the most intensive BP1 immunoreactivity. Cells with BP1 expression showed a substantially higher proliferation rate than morphologically comparable cells without BP1 expression.

These findings suggest that BP1 is an important upstream factor in an oncogenic pathway and that the expresssion of BP1 may reliably reflect or directly contribute to tumor progression and/or invasion.

More specifically, consecutive sections were made from formalin-fixed, paraffin-embedded normal prostate (n=5) and prostate tumors (n=50) with co-existing normal, hyperplastic, and neoplastic tissues. The first and last sections from each case were stained with hematoxylin and eosin for morphological classification, based upon published criteria.

A polyclonal antibody against the BP1 peptide was made from rabbit, as previously described. Monoclonal antibodies against human Ki-67 antigen (clone MIB-1), proliferating cell nuclear antigen (PCNA), a biotinylated secondary antibody, the ABC detection kits (avidin-peroxidase or avidin-alkaline-phosphate conjugated), the chromogen diaminobenzidine (DAB) kit, and normal serum were purchase from Vector (Burlingame, Calif.). The A-red chromogen kit was obtained from Zymed (San Francisco, Calif.). A background blocking solution was from Cell Marque (Hot Springs, Ark.). Other immunostaining related reagents, including the antigen retrieval solution, Tris-buffered saline (TBS), and hematoxylin and eosin were purchased from Biocare-Medical, LLC (Walnut, Calif.). Before application to experimental cases, several methods were used to verify the specificity of the BP1 immunostaining. First, the BP1 antibody was pre-absorbed with the purified BP1 protein at different concentrations before the incubation with the sections. Second, different concentrations of BP1 antibody were incubated with consecutive sections in different prostate lesions from different patients. Third, tissue culture cells with different levels of BP1 expression were grown on cover glasses and were immunostained with the BP1 antibody. Fourth, the primary antibody was substituted with phosphate-buffered saline (PBS) or normal serum, or the secondary antibody was omitted from the immunostaining sequence. The intensity of BP1 immunostaining decreased with decreased antibody concentration. The substitution of the primary antibody with PBS or normal serum, or the omission of the secondary antibody from the immunostaining sequence resulted in total loss of BP1 positive immunostaining.

In addition, several methods were used to optimize the immunostaining. Consecutive sections from 5 different cases were subject to different pre-treatments, including microwave irradiation, pressure-cooker incubation, an overnight incubation at about 70° C., a two hour incubation at 80° C., and a routine deparrafin process without other treatment, and immunostained with the same antibody solution and the same immunostaining procedure, to compare the sub-cellular localization, positive cell number, and the intensity of the BP1 staining. Furthermore, consecutive sections were incubated with the same monoclonal antibody but at different temperatures (4° C. overnight, room temperature overnight, and 37° C. for two hours). Finally, two immediate adjacent sections pre-incubated with the same primary antibody were detected with two different detection systems, peroxidase with DAB and alkaline phosphatase with AP-red. After a series of tests, the combination of pre-treatment at 80° C. for two hours, an overnight incubation of the primary antibody at 4° C., and detection with avidin-alkaline phosphatase complex was found to yield the best results. This approach was consequently used for immunostaining of experimental slides. Immunhistochemical staining was carried out using the ABC method. Ducts and acini lined by over 40 epithelial cells were examined for BP1 expression. A given cell was considered BP1 positive if distinct chromogen was consistently detectable in its cytoplasm or nucleus in at least two duplicates of the same immunostaining procedure. A given duct or acinus was considered BP1 positive if more than 5% of its constitute cells showed distinct BP1 immunoreactivity. In cases containing multiple histological components, the BP1 expression status in each component was separately evaluated and recorded. The frequency of BP1 expression among normal, hyperplastic, and neoplastic cells was statistically compared with the Student-t Test.

Distinct BP1 immunostaining was seen predominantly in epithelial cells, and occasionally seen in some white blood cells and smooth muscle cells. No distinct BP1 immunostaining was detected in fibroblasts, adipocytes, and collagen fibers. BP1 immunoreactivity was predominantly in the cytoplasm, and barely seen in the nuclei of cells.

Of the 5 normal controls, 3 were completely devoid of BP1 immunostaining in the epithelium, and 2 contained BP1 positive cell clusters, which accounted for less than 5% of the total cell population, so did not meet the criteria for BP1 positivity.

Of the 50 cases containing co-existing normal, hyperplastic, and neoplastic tissue components, the prevalence of BP1 positive cases increased significantly as the histologic type became more progressive (p<0.0001). Of the normal tissue component, 10 (20%) cases were BP1 positive. Of the hyperplastic component, 21 (42%) cases were BP1 positive. Of the neoplastic component, 40 (80%) were BP1 positive.

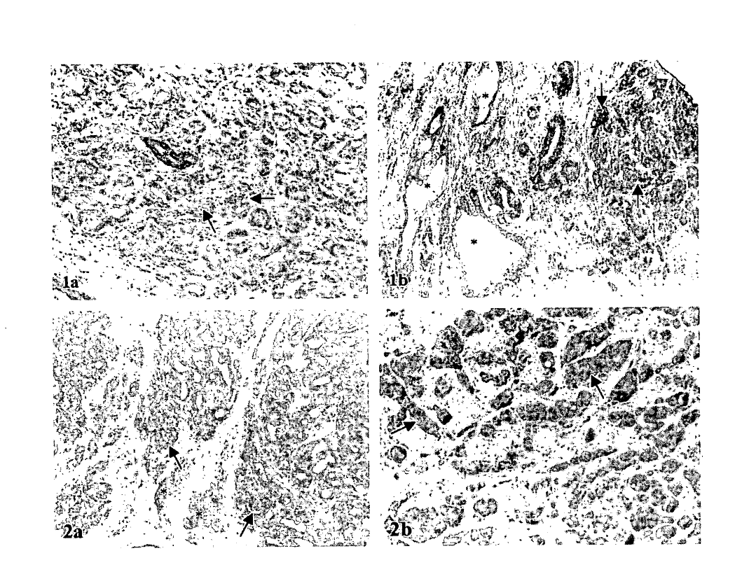

There was also a substantial increase in the number of BP1 positive cells and intensity of BP1 immunostaining as histological types became more aggressive. In normal and hyperplastic tissue components, BP1 positive acini and/or ducts were generally distributed as clusters with a defined boundary with their adjacent BP1 negative counterparts (FIG. 1A). In some cases, BP1 positive and negative cells co-existed in different sites of the same duct (FIG. 1B). The BP1 positive cells in both normal and hyperplastic components generally accounted for no more than 30% of the total cell population. In the neoplastic component, cancer cells were generally uniformly BP1 positive (FIG. 2A). Cancer cells also consistently showed the highest intensity of BP1 immunostaining, compared to their normal and hyperplastic counterparts in each of the cases examined (FIG. 2B).

To monitor BP1 expression associated with cell proliferation, sections from morphologically similar hyperplastic and neoplastic lesions with (n=5) and without (n=5) BP1 expression were double immunostained for BP1 and Ki-67. The cell proliferation rate in 3-5 morphologically similar ducts in each case was calculated, and the rates among each category were averaged and statistically compared. Compared to their morphologically similar counterparts without BP1 expression, BP1 positive cells showed a significantly higher (p>0.05) proliferation rate, 8.8% versus 3.0%.

Our current study reveals that the frequency and intensity of BP1 positive immunostaining increase with the progression of the tumor stages (normal->hyperplasia->neoplasia), from a few randomly distributed BP1 positive cell clusters in normal controls to the vast majority of cells in 80% of neoplastic lesions showing distinct BP1 immunoreactivity. Compared to normal and hyperplastic tissue components, the neoplastic component consistently showed the highest number of BP1 positive cells, and the highest intensity of BP1 immunoreactivity. The overall expression of BP1 in prostate tumors is very similar to that in breast tumors.

The preferential cytoplasmic localization of BP1 was at first unexpected since BP1 can act as a transcription factor. However, it is known that homeotic proteins can be transported to the cytoplasm and secreted, then taken up by other cells.

Not only is BP1 aberrantly expressed in prostate and breast tumors, it is also expressed in 63% of the bone marrow (BM) of acute myeloid leukemia (AML) patients, including 81% of pediatric and 47% of adult patients, as well as in 32% of pediatric T-cell acute lymphocytic leukemia patients. In contrast, BP1 mRNA is barely if at all detectable in normal BM or PHA-stimulated T cells (U.S. Pat. No. 6,416,956, incorporated herein by reference in its entirety).

Together, our current and past findings suggest that BP1 may be an important upstream factor in an oncogenic pathway. While the specific role(s) of BP1 in tumor development and progression has not been defined, it seems to be specifically involved in blocking the normal process of a programmed cell death (apoptosis), facilitating the formation and expansion of a biologically more aggressive cell clone, since:

Our previous studies have shown that ectopic expression of BP1 in leukemia cell line K562 substantially increased their clonogenicity, suggesting that BP1 is capable of sustaining or facilitating deregulated cell proliferation, which has been regarded as a direct cause of malignancy.

Abrogation of BP1 expression in K562 cells causes apoptosis (P.E.B, unpublished data). Both prostate and breast cancer cells expressing BP1 have a significantly higher cell proliferation index, compared to their morphologically comparable counterparts without BP1 expression.

BP1 positive cells are generally distributed as distinct clusters of foci with a well-defined boundary to adjacent BP1 negative cell clusters (see FIG. 1), or as a well-defined ductal segment that is connected to BP1 negative cells, consistent with the typical feature of clonal proliferation and expansion.

In preliminary studies, injection into the fat pads of nude mice using breast cancer cell lines which overexpress BP1 caused increased numbers of breast tumors compared with controls (P.E.B. and B Vonderhaar, unpublished data).

Together, these findings suggest that BP1 is an important upstream factor in an oncogenic pathway, and that expression of BP1 may reliably reflect or directly contribute to tumor progression and/or invasion.

Exemplary Methods.

As exemplified above, the present invention is useful in the detection of hyperplastic and/or neoplastic cells from a sample derived from prostate tissue. This utility provides methods, generally described below, for (i) diagnosis of hyperplastic and/or neoplastic cells in a prostate tissue sample; (ii) methods for determining the aggressiveness and/or invasiveness of such cells, and; (iii) methods for monitoring therapeutic treatments designed to effectively treat such cells.

Diagnosis of Hyperplastic and/or Neoplastic Prostate Cancer Cells.

The methods for diagnosing hyperplastic and/or neoplastic cells under the invention comprise:

providing a prostate tissue or cell sample (see below), containing one or more cells;

contacting this tissue or cell sample with an anti-BP1 antibody or antibody fragment under conditions whereby the antibody or fragment can bind to BP1;

incubating the tissue or cell sample with the anti-BP1 antibody for a time and under conditions sufficient for the antibody to bind to BP1 protein in the tissue sample or cell sample; and,

determining whether the antibody has bound to one or more of the cells in the tissue sample or cell sample.

This method is exemplified above for a tissue sample, and can readily be applied to a cell sample by those skilled in the art.

The sample may be provided, for example, in the form of a tissue slice, or may be independent cells. Tissue samples under this method of the invention are preferred to be prostate tissue samples, or tissue in the proximate vicinity of prostate tissue. Cell samples under the invention may be any cell sample that includes, or is suspected to include, the presence of cells having a prostate origin.

The antibody used in the practice of the invention may be monoclonal or polyclonal. The antibody may be intact or may be a BP1-binding fragment thereof, for example, an Fv fragment, and may be a natural antibody, a humanized or otherwise engineered antibody, with the stipulation that it specifically bind the BP1 protein.

For immunochemical staining, the staining compound may comprise, for example, a colorimetric, fluorimetric, phosphorimetric, or radiometric moiety. Preferably, the staining compound is colorimetric or fluorimetric, and most preferably calorimetric.

Reagents for use in the diagnostic methods of the invention may be standard reagents, optionally purchased from one or more suppliers, as is known in the art. The antibody or other reagents may be pre-treated prior to contacting with the cells. Such pre-treatment includes heating regimes at suitable temperature, microwaving, and the like. Incubation may be also be carried out at suitable temperature and for suitable times, as exemplified above, to permit specific binding of the antibody to the BP1 protein.

Determining the Aggressiveness and/or Invasiveness of Prostate Cancer Cells

The invention also provides for a method of diagnosing the aggressiveness and/or invasiveness of prostate cancer cells, following the steps as provided above, and including the additional step:

comparing the amount and/or degree of antibody staining of a BP1 positive cell(s) in the tissue sample or cell sample to the staining of normal prostate and/or other cells in the sample, whereby the intensity of the staining is positively correlated to the aggressiveness and/or invasiveness of the cancerous cell(s).

Monitoring Therapeutic Treatment of Prostate Cancer

The invention further advantageously provides a means for monitoring the effectiveness of a therapeutic regimen over the course of treatment for prostate cancer. This method involves the steps given above (with or without step (5)) at some initial time in the therapy (e.g., at the beginning of the therapy). At a later time, the method of the invention is again performed to determine the effectiveness of the therapeutic regimen over the intervening period, as measured by number of BP1 positive cells and/or change in the intensity of the stain. An effective therapy shows a lower number of cancerous cells and/or less intense staining at the later time. An ineffective therapy results in either no substantial change, or an increase, in the numbers of cells and/or intensity of staining at the later time.

The method of the invention can be performed a third or more times, each time comparing the newly-obtained results with those previously obtained. Where the therapeutic regimen was initially effective, as described above, there will be a decrease in the number of BP1-positive cells and/or intensity of staining early in the treatment. Where the treatment later becomes less effective, the number of BP1-positive cells and/or the staining of the cells will again increase over the previous test(s). Where appropriate, a new therapeutic regimen may be administered, and it's effectiveness determined/and monitored using the methods of the invention.

Diagnostic Kits.

The invention further comprises diagnostic kits containing anti-BP1 antibody and, if desired, one or more immunostaining reagents, suitably packaged for storage and/or transport. The kit may additionally contain control immunostaining reagents. The immunochemical reagents may include those useful for tagging, staining, developing, etc., to generate a detectable signal. Additional reagents, such a buffering salts or solutions, as well as devices, for example, for dispensing of the reagents, may also optionally be included in the kits.

It is understood that various other modifications will be apparent to and can readily be made by those skilled in the art without departing from the scope and spirit of this invention. Accordingly, it is not intended that the scope of the invention be limited to the description set forth above, but rather be construed as encompassing all of the features of patentable novelty which reside in the present invention, including all features which would be treated as equivalents thereof by those skilled in the art to which the invention pertains.

We claim:

1. A method for the diagnosis of hyperplastic and/or neoplastic prostate cancer cells, comprising:
providing a prostate tissue or cell sample containing one or more cells;
contacting said tissue or cell sample with an anti-BP1 antibody or antibody fragment under conditions whereby the antibody or fragment binds to BP1;
incubating said tissue or cell sample with the anti-BP1 antibody for a time and under conditions sufficient for the antibody to bind to BP1 protein in the tissue sample or cell sample; and,
determining whether the antibody has bound to one or more of the cells in the tissue sample or cell sample.

2. The method of claim 1, further comprising the step of determining the aggressiveness and/or invasiveness of prostate cancer cells by comparing the amount and/or degree of antibody staining of a BP1 positive cell(s) in said tissue sample or cell sample to the staining of normal prostate tissue and/or other cells in the sample, whereby the intensity of the staining is positively correlated to the aggressiveness and/or invasiveness of the cancerous cell(s).

3. A method for monitoring therapeutic treatment of prostate cancer, comprising:
at a first time, providing a prostate tissue or cell sample containing one or more cells;
contacting said tissue or cell sample with an anti-BP1 antibody or antibody fragment under conditions whereby the antibody or fragment binds to BP1;
incubating said tissue or cell sample with the anti-BP1 antibody for a time and under conditions sufficient for the antibody to bind to BP1 protein in the tissue sample or cell sample;
determining whether the antibody has bound to one or more of the cells in the tissue sample or cell sample; and,
repeating the contacting, incubating and determining steps at a later time and comparing the level of BP1 antibody staining and/or the number of BP1-positive cells at said later time with the staining level and/or number of positive cells at said first time.

4. A diagnostic kit for the detection of prostate cancer, said kit comprising:
an anti-BP1 antibody or BP1-binding fragment thereof; and
an immunostaining reagent that reacts with said anti-BP1 antibody or fragment.
